(19) United States
(12) Patent Application Publication
(10) Pub. No.: US 2010/0023102 A1
(43) Pub. Date: Jan. 28, 2010

(54) Layered Electrode Array and Cable

(75) Inventor: Arjen Spruit, Victoria (CA)

Correspondence Address: QUARLES & BRADY LLP, ONE SOUTH CHURCH AVENUE, SUITE 1700, TUCSON, AZ, 85701-1621, US

(73) Assignee: Med El Elektro-Medizinische Gerate Gesellschaft m.b.H, Innsbruck (AT)

(21) Appl. No.: 12/375,394

(22) PCT Filed: Jul. 27, 2007
(86) PCT No.: PCT/CA2007/001333

§ 371 Date: Apr. 21, 2009

Related U.S. Application Data
(60) Provisional application No. 60/820,662, filed on Jul. 28, 2006.

(51) Int. Cl. A61N 1/05; H01R 43/00

(52) U.S. Cl. 607/116; 29/825

(57) ABSTRACT

A method of fabricating a neurostimulation circuit is disclosed. According to the present invention, individual implantable assembly layers are cut by a laser or by mechanical means, and then stacked together, thereby providing a more efficient manufacturing method for manufacturing high density implantable electrode arrays and cables. In the invention, the separate implantable assembly layers can be melted and conglomerated to form a neurostimulation circuit in which the conductors and terminal pads are encapsulated within a continuous polymer insulating film.

### FIELD OF THE INVENTION

The present invention relates to an implantable medical assembly having a biologically compatible film within which at least one electrode and at least one conduction wire connected to the electrode to provide the stimulation signal for human nerves are embedded, and more particularly to the design of the conduction wires embedded in the biologically compatible film. More particularly, the present invention relates to a method of forming electrode arrays, such as arrays for sensors, including biosensors, and implantable devices, such as an implantable recording or stimulating electrodes or leads for use in the body.

### BACKGROUND OF THE INVENTION

For several years, researchers have been attempting to establish communications through living neurons. It is now well known that electrical stimulation of certain nerves and certain regions of the brain can be used to convey information which can no longer be provided by a person's own eyes or ears, stimulate paralyzed muscles, stimulate autonomic nerves, control bladder function, pace the heart, or control prosthetic limbs, to name a few of the growing list of applications.

To accomplish this, an electrical connection must be established between a source of electrical stimulation and target neurons. Such connection must be made via extremely small electrodes, in order to isolate electrical currents within very small regions of living tissue. These small electrodes can be placed in very close proximity to the target nerve cells, and electrical current provided by the stimulation source can then be directly injected into the nerves. To limit the mechanical trauma caused by insertion and chronic presence of electrode structures, the entire electrode structure and associated conduction wires must be as small as possible, consistent with the required ability to conduct electrical energy, and must be made of materials which will neither react with the living body or be damaged by the corrosive environment of the body.

Implanted electrodes and the conduction wires connected to them must be very effectively insulated, because of the very small voltages and currents being utilized.

Further, many neurostimulation devices require a large number of electrodes placed in close proximity to neural structures to facilitate effective stimulation. In addition, neurostimulation devices require a hermetic housing where the stimulation signals and power are generated. Because the housing is large compared to the stimulation electrodes, the electronics package may need to be surgically placed in a location remote from the stimulation site.

It is therefore required that there be a conductor cable connecting the electronics housing to the electrodes. With the trend towards ever-increasing numbers of electrodes, conduction wires with ever-increasing numbers of individual channels are needed and thus ever-increasing numbers of conduction pathways.

Because the conduction wires are located within the body, they must be made to withstand millions of micro-movements to facilitate continuous operation over the long-term.

Also, conduction wires and electrodes must be constructed of bioresistive, biocompatible materials that do not cause adverse tissue reactions and that allow the structure to endure and function within the hostile electrolytic environment of the human body.

Neurostimulation devices should also be reliably producible and relatively inexpensive to fabricate.

Platinum electrodes and conduction wires can be conveniently formed using standard techniques such as laser cutting of platinum foil or chemical etching of platinum foil (see for example, R. P. Frankenthal, et. al., Journal of Electrochemical Society, 703(123), 1976).

Alternatively, a well-known photolithographic method whereby a thin coating of platinum is vacuum deposited or sputtered through a photomask, with subsequent electroplating to increase the thickness of the platinum can be used. For example, M. Sonn, et al., (Medical and Biological Engineering, pp. 778-790, November 1974) and M. Sonn (A Raytheon Company Publication PB-219 466, available from the U.S. National Information Service, U.S. Department of Commerce) used, amongst other substrates, the polyfluorocarbon FEP (fluorinated ethylene propylene) as a substrate onto which platinum conductors and electrodes were sputtered, with the electrode and conductor patterns defined by photolithographic etching means.

G. M. Clark, et al., (Journal of Laryngology and Otology, Vol. XC/No. 7, p623-627, 1976) describe a multi-electrode ribbon-array using a thin 0.1 μm layer of RF sputtered platinum onto FEP, subsequently insulated with FEP, and the electrode stimulating areas exposed. An array of platinum can be made to adhere to an FEP substrate insulated with additional FEP, and exposed at electrode stimulating areas. Bending tests on the array indicate that it is both flexible and strong.

H. D. Mercer, et al. (IEEE Transactions on Biomedical Engineering, Vol. BME-25, No. 6, November 1978) describe a planar lithographic technique for fabrication of a microelectrode array for a cochlear prosthesis using a sputtered platinum layer with thin molybdenum and tungsten substrates.

G. A. May et al. (IEEE Transactions on Electron Devices, Vol. ED-26, No. 12, December 1979) describe an eight-channel tantalum-on-sapphire multi-electrode array design using planar photolithography. The sapphire substrate was chosen for its electrical and mechanical properties, tantalum was applied as the conductor metal, and platinum was applied as the stimulation electrode material.

C. R. Pon, et al. (Ann. Otol. Rhinol. Laryngol. 98(6) 66-71, 1989) attempted to form a standard “ring type design” electrode array by using planar photolithography to define the electrode features, RF sputtering platinum onto a polyimide substrate, rolling up the film substrate into a cylindrical shape, and filling it with medical grade silicone rubber.

J. L. Parker et al., in U.S. Pat. No. 5,720,099, describe a photolithographic technique for fabricating an elongated electrode array assembly by first depositing pads on a sacrificial layer, adding wires to the pads (such that the wires are self-supporting when the photoresist mask is removed), then embedding the wires and pads in an insulating material such as silicone elastomer, and finally removing the sacrificial layer. Importantly, a photolithographic process is used to produce the electrode assembly using a sacrificial layer as the initial base.

Those familiar with the art of photolithography and electrochemical deposition processes used in the microelectronics industry will appreciate that there are a number of well established technologies for forming micro-patterns of metals and polymer encapsulation thereof.

Manrique Rodr Guez, Manuel et al, in European Patent No 1,574,181 A1, describe an electrode-bearing guide, a cochlear implant comprising the guide and the production method thereof. The electrode carrier guide is formed by the superposition of a series of basic cells. In this invention, an adhesive biocompatible material which is arranged between the base layer and electrically conducting layer is employed for enhancing adherence.

To better understand and appreciate the present invention, it will be helpful to briefly review an existing implantable medical assembly that is representative of other tissue-stimulating systems. An implantable medical assembly of the type currently fabricated is described in U.S. Pat. No. 6,374,143 B1, and illustrated in FIGS. 1 to 4. FIG. 1 is an implantable medical assembly having biologically compatible film within which electrodes and conduction wires connected to the electrodes provide the stimulation signal for human nerves according to prior art. A polymer film 10 has three electrodes (1, 2 and 3) and one conduction wire 8 per electrode, disposed therein. The electrodes 1, 2, and 3 and conduction wires 8 can be fabricated from a biologically compatible and inert metal such as platinum, tantalum, rhodium, rhenium, iridium or alloys thereof, or a combination of two or more alloys and/or metal layers thereof.

The electrodes 1, 2, and 3 and the conduction wires 8 are held in place by an inert film material 10, preferentially the polyfluorocarbon FEP, although any biologically inert, high dielectric constant flexible material may be suitable. As shown in FIG. 1, each conduction wire 8 is connected to each electrode to provide a signal from the stimulator to the human nerves. Those skilled in the art will note that a myriad of possible configurations for the electrodes are possible according to neural shapes, sizes and positions.

The conduction wires 8 have an approximate width of 10-100 μm and an approximate thickness of 2-50 μm. The thickness of the encapsulating film 10 is about 20-100 μm.

Furthermore, numerous studies have been conducted to identify the biocompatibility of various implant materials (see for example “Biocompatibility of Clinical Implant Materials”, Volumes 1 and 2, edited by David F. Williams, published by CRC Press, Inc., Boca Raton, Fla., USA). Some commonly used biomaterials, well known to those skilled in the art, include titanium (and some alloys thereof), platinum, tantalum, niobium, iridium, gold, some ceramics (such as alumina), certain carbon materials, some silicones, and polymers such as the fluorocarbons FEP, PTFE, PVDF, PFA, PCTFE, ECTFE, ETFE and MFA (a copolymer of TFE and PVE), polyethylenes, polypropylenes, polyamides, polyimides and liquid crystal polymers.

FIG. 2 is a cross-sectional view of section ‘A-A’ of FIG. 1 showing an embedded metal electrode 1 and three conduction wires. The electrode is exposed to a human nerve to transfer the stimulation signals from the stimulation source via the conductor 8.

FIG. 3 is a planar view to illustrate where to fold-in and fold-out an implantable medical assembly according to prior art. FIG. 4 is a perspective view showing the film being folded over along the folding-in and folding-out lines L1, L2 and L3. To make a suitable shape and size for a neural stimulation implant assembly, such as a cochlear implant, the implantable medical assembly needs to be folded along the virtual in-folding and out-folding lines L1, L2 and L3 established by the manufacturer.

When folding the medical assembly, careful handling of the assembly is required. For example, one stimulation implant may need to incorporate multiple folds or more without impairing the structure of the implantable medical assembly.

In prior art shown from FIG. 1 to FIG. 4, the conduction wires 8 may be broken or may be easily fractured during the folding process. The implantable medical assembly requires discrete electrical continuity of the individual conduction wires and electrodes to ensure signal transfer between target nerves and the implant housing wherein electronic circuits to control the nerves reside. If only one conduction wire is fractured, partial or total malfunction of the implant may result.

Electrode arrays and lead components of implantable neurostimulation devices, such as cochlear implants, are still manufactured using labor intensive manual procedures. In such devices, size needs to be minimized to ensure that the implant and the implantation procedure are only minimally invasive. As a result, in such instances, the electronic wiring and connections need also to be relatively very small. As such, manufacturing such devices to ensure that they are reliable and sturdy is a specialized craft, and requires much time and expense. Ensuring that the wiring and connections of the various components of the systems occurs correctly is often the most expensive and labor intensive aspect of the manufacturing process and can result in high manufacturing costs particularly if such devices need to be specifically hand made. While the manual method has proven relatively successful to date, it has an intensive labor component and hence is a relatively expensive process.

With implanted devices and miniaturization becoming more common, there is an increasing need to provide electrode arrays and lead components for such systems that are both simple and reliable to fabricate. The present invention is directed to a new method of forming such components that addresses at least some of the problems with prior art processes.

As a result of the need to increase the miniaturization of such neurostimulation devices, a wide range of techniques has been developed to create patterned components which would be too difficult or impossible to create by hand design and satisfy the high volume required to meet industry demands. This is particularly the case in the field of medical implants and electrical devices that are implanted in the body to perform specific tasks. Such devices may include: stimulating devices such as pacemakers, cochlear implants, FES stimulators; recording devices such as neural activity sensors and the like; implantable cables which may be used to connect implantable devices to other implantable devices or stimulating/sensing devices; diagnostic devices capable of carrying out in-vivo analysis of body parameters; and other types of implantable devices not yet contemplated.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### SUMMARY OF THE INVENTION

In view of the above-mentioned disadvantages of the prior art, it is an objective of the present invention to provide an implantable medical assembly for various neurostimulation systems such as cochlear implants.

A further objective of the invention is to provide an implantable medical assembly which can be reliably implanted with long term stability.

A still further objective is to provide an implantable medical assembly which has more stable mechanical and electrical characteristics.

In view of the foregoing, another objective of the present invention is to provide an implantable medical assembly, which has an improved manufacturing process, compared to the prior art manufacturing processes.

A yet further objective of the present invention is to provide an implantable medical assembly which is easier to manufacture.

In accordance with the present invention, an implantable medical assembly comprises a biologically compatible film, at least one electrode on the film, and at least one wire on the film being continuous with the electrode to provide a stimulation signal, wherein the wires have a photolithographically defined straight or undulated shape.

In the present invention, first, on the substrate, the electrodes and the conductor wires made of platinum or other noble metal are deposited through an electrodeposition process. Then, the first FEP film is laminated to cover all the substrate including the electrodes and the conductors. Next, the substrate is removed, and then another FEP film is deposited to cover the remaining structure, thus embedding the electrodes and the conductor wires within the FEP film. Then, the electrodes can be exposed as shown in FIG. 2. This whole process may be performed through a photolithographic method using the prior arts already mentioned.

Preferably, in accordance with the present invention, an implantable medical assembly comprises a biologically compatible film, at least one electrode within the film, at least one wire within the film and being connected to the electrode to provide a stimulation signal. In the implantable medical assembly, the wire has a straight or undulated shape. Further, the medical assembly is cut according to the cutting line(s) by a laser cutting or a traditional knife, is then corrugated, and finally encased within an elastomer such as a silicone. Preferably, in accordance with the present invention, two or more implantable medical assemblies each of which consists of one biologically compatible film, at least one electrode within the film and at least one wire within the film, can be stacked continuously. Also, one implantable medical assembly, which consists of a biologically compatible film, at least one electrode and at least one wire within the film, can be folded and stacked. Further, the medical assembly can be encased within an elastomer such as silicone.

These circuit structures having fine line circuit patterns can be difficult to form in an efficient and cost effective manner. For example, a typical flexible circuit structure includes one or more flexible fluoropolymer films with one or more conductive patterns. Forming conductive patterns directly on flexible fluoropolymer film is difficult, because the film is flimsy and thin.

As already mentioned, this invention utilizes photolithographic technology. To produce a narrow cable containing a large number of conduction wires requires that the wires be spaced very close together. According to one development of the invention, the process for building up multiple layers to incorporate a large number of conductors into a narrow thin cable is proposed.

Implantable nerve stimulators, by utilizing a fluoropolymer film as the insulating material, can typically occupy less space than conventional nerve stimulators that use silicone as a carrier for the electrode array. The reduced space provided by fluoropolymer film structures make them especially suitable for use in small medical products such as neurostimulation devices used to help restore or maintain some degree of lost sensory or motor function in neurologically impaired individuals. In addition, implantable nerve stimulator structures utilizing fluoropolymers can be highly reliable because of the excellent insulating capacity of the material, its lack of chemical or biochemical reactivity and its mechanical stability.

Furthermore, according to the present invention, instead of using a folding process, the medical stimulation layers can be superposed in a stacked shape.

Other aspects of the invention will be appreciated by reference to the detailed description of the invention and to the claims.

### BRIEF DESCRIPTION OF THE DRAWINGS

The preferred and alternative embodiments of the invention will be described with reference to the accompanying drawings, in which:

FIG. 1 is a planar view of an implantable medical assembly having biologically compatible film within which electrodes and conduction wires, connected to the electrodes, provide a pathway for stimulation signals to reach human nerves according to prior art;

FIG. 2 is a cross-sectional view of section A-A of FIG. 1 showing some embedded metal electrodes and conduction wires according to FIG. 1;

FIG. 3 is a planar view illustrating how to fold-in and fold-out an implantable medical assembly according to prior art;

FIG. 4 is a perspective view showing the film being folded over along the fold-in and fold-out lines;

FIG. 5 shows a planar view of a preferred embodiment of an implantable medical assembly with cutting lines according to the present invention;

FIG. 6A shows a planar view of an implantable medical assembly with multiple layers which have conduction wires connected to corresponding electrodes according to the present invention;

FIG. 6B shows a side view of an implantable medical assembly with multiple layers after heat treatment according to FIG. 6A;

FIG. 7 shows a schematic view of the implantable medical device having an overall corrugated shape according to the present invention;

FIG. 8A shows a schematic view of the implantable medical device having an overall corrugated shape encased with elastomer such as silicone according to the present invention;

FIG. 8B shows a side view of the implantable medical device having an elastomer such as silicone bonded onto the underside of the electrode portion of the assembly;

FIG. 9A shows a perspective view of the implantable medical assembly having its terminal end ready for connection to a control device; and

FIG. 9B shows a perspective view of the implantable medical assembly having double-sided terminal end ready for connection to a control device.

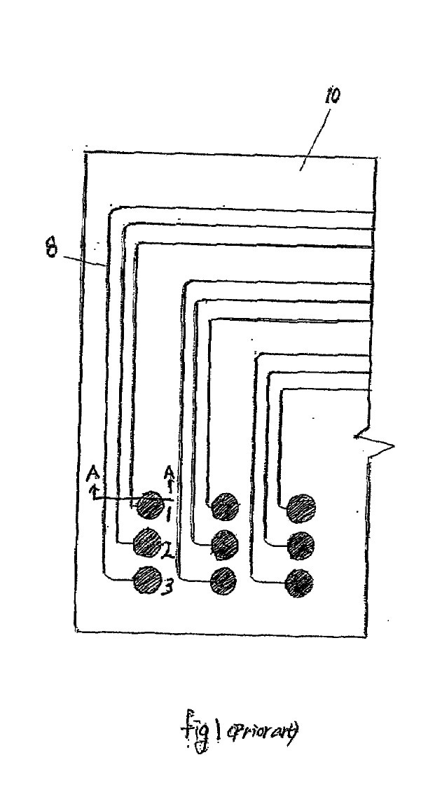

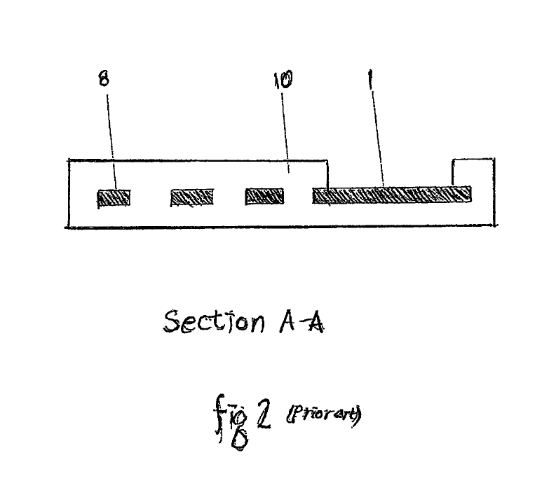

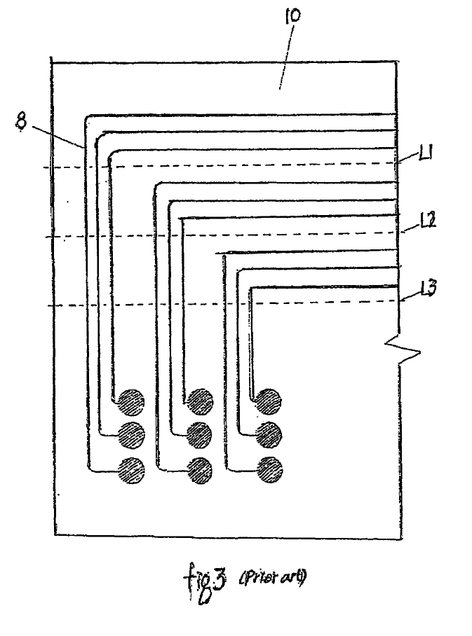

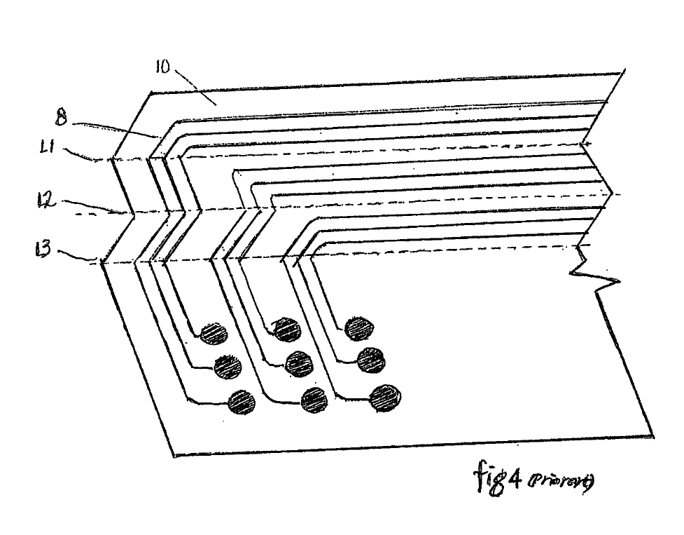

### DETAILED DESCRIPTION OF THE PREFERRED AND ALTERNATIVE EMBODIMENTS OF THE INVENTION

The following describes the best mode presently contemplated for carrying out the invention. This description is not to be taken in a limiting sense, but is made merely for describing the general principles of the invention. The scope of the invention should be determined with reference to the claims.

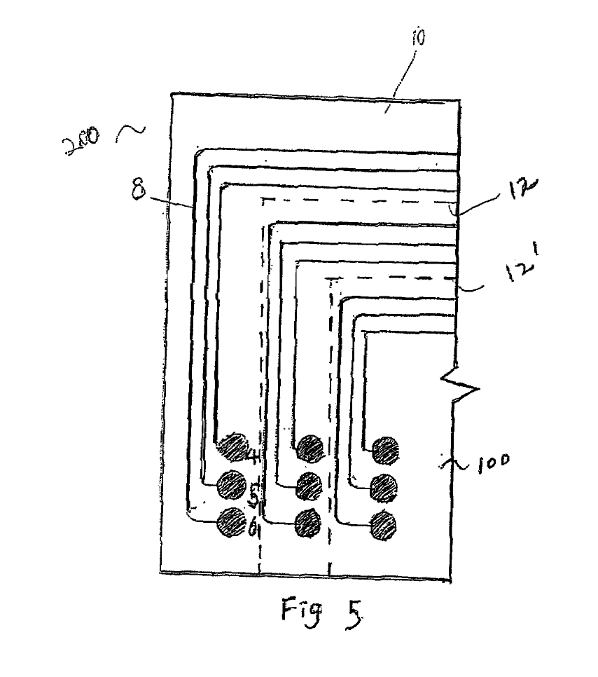

FIG. 5 shows a planar view of a preferred embodiment of an implantable medical assembly with electrodes and conduction wires according to the present invention. The aforementioned implantable medical assembly is designed to carry electrical signals from the housing that contains the electrical stimulator to the electrodes of an implantable nerve stimulation device for the purpose of safely and reliably stimulating human nerves. According to an implantable medical assembly 200, shown in FIG. 5, conduction wires 8 continuous with electrodes 4, 5 and 6 are embedded within a suitable biocompatible material 100, such as FEP film. The wires 8 and electrodes 4, 5, and 6 are formed using well-known photolithographic and electrochemical deposition processes and encapsulated within biocompatible material using established polymer encapsulation techniques. The insulating FEP film is removed from the electrode surfaces by laser ablation or mechanical means. According to the present invention, cutting lines 12, 12′ on the film can be cut using a laser or a traditional knife to form a series of separate film components or layers.

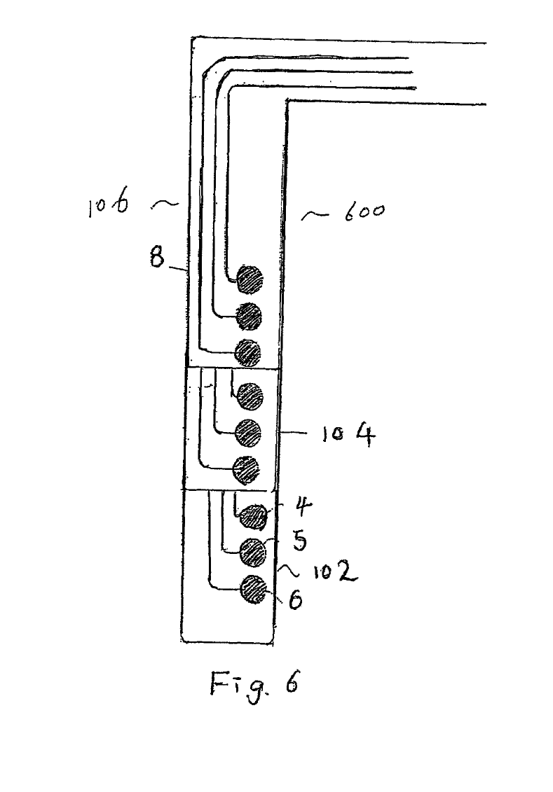

FIG. 6 shows a planar view of an implantable medical assembly with multiple layers, each having one or more conduction wires continuous with their corresponding electrodes according the present invention.

Two or more implantable assembly layers 102, 104, and 106 are stacked in an offset fashion, thereby allowing all of the electrodes to be exposed. Each layer 102, 104, and 106 composing the electrode and conductors has the same width and thickness, but may or may not have the same length. The layers are aligned on top of each other and are superposed in a stepped shape. This stacking process can eliminate slow and expensive manufacturing process such as a folding process discussed above. The implantable assembly layers 102, 104, and 106 can be consolidated using heat and pressure.

The separate implantable assembly layers 102, 104 and 106 can then be melted and conglomerated into a single assembly. Also the separate assembly layers may be adhered together by using medical grade adhesive between each assembly layer.

The implantable assembly layers 102, 104, and 106 shown in FIG. 6 are conglomerated into a single continuous film, having no boundaries between layers, by heat treatment. Preferably, a layer of silicone can be over molded to form a constrained shape. This silicone layer can be added to part of the assembly structure or molded over the whole assembly.

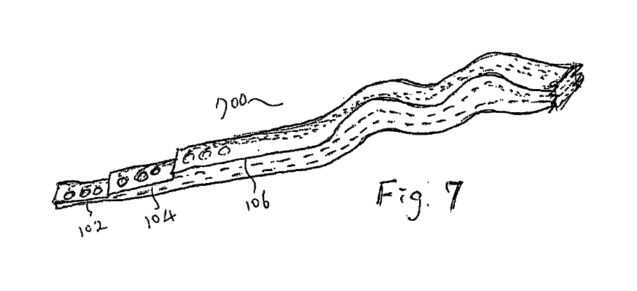

FIG. 7 shows a perspective view of the implantable medical assembly with multiple layers having an overall corrugated shape according to the present invention. This assembly 700 may be applied to a cochlear implant or other nerve stimulating implant, wherein an extensible cable or lead is required. Further, this assembly 700 may be used as a connection cable to the electronics housing containing the stimulation source, or as a connection cable between two electronics housings. To increase the expandability and elasticity of the implantable medical assembly 700, after a predetermined stacking process, the implantable medical assembly or a portion thereof is molded to have the corrugated shape as shown in FIG. 7. Therefore, the implantable medical assembly 700 can readily be expanded or contracted.

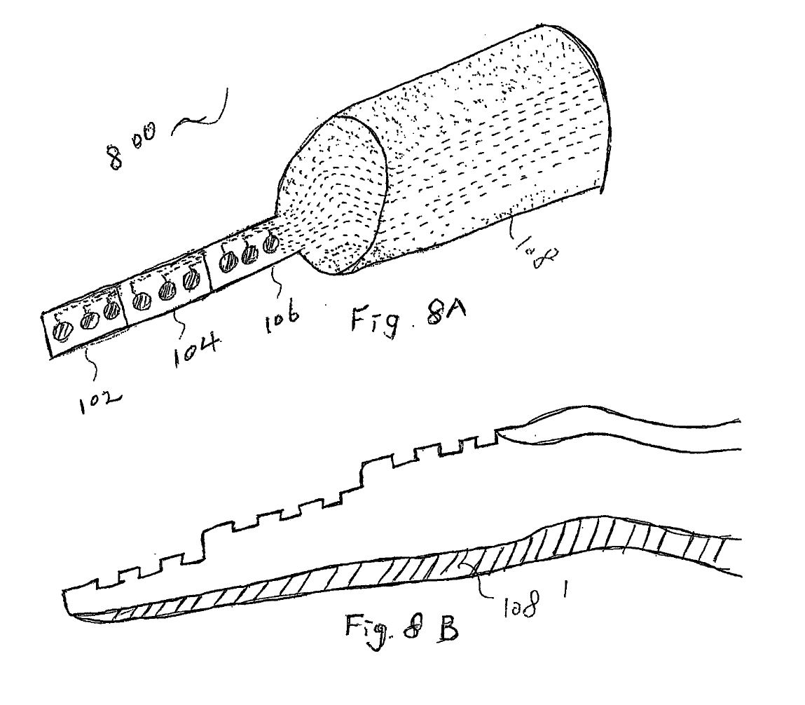

FIG. 8A shows a perspective view of the implantable medical assembly having an overall corrugated shape encased with elastomer such as silicone according to the present invention. The implantable medical assembly 800 is encased with a biocompatible elastomer 108 such as silicone to protect the overall implantable medical assembly 800 and to facilitate ease of handling of the assembly during implantation according to the present invention. The cross sectional configuration of the silicone encapsulant may be circular, square, rectangular or any appropriate shape as dictated by the application of the device.

FIG. 8B shows a side view of an alternative embodiment of the assembly in which an elastomer 108′ such as silicone is embedded onto the underside of electrode portion of the assembly to enhance the implantability of the assembly.

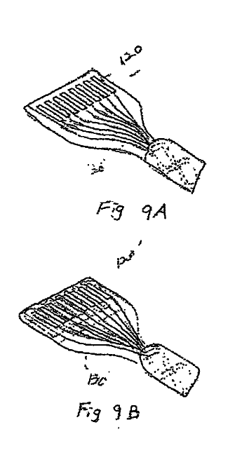

FIG. 9A shows a perspective view of the implantable medical assembly having a terminal end which can be connected to a test device or stimulation source (not shown). As shown in FIG. 9A, this end terminal 120 will be easily connected to a test device or stimulation source (not shown) according to the number of channels or its application. The terminal 120 can be made of the same material used for the rest of the assembly including the electrodes and the conduction wires. Implantable neurostimulation devices comprise an electronic control device coupled with an electrode array/cable system that conducts current to the target site. The electrode array/cable systems are normally constructed of one or more conductor wires which, at one end are located the stimulating electrodes (the electrode array) and at the other, a connection element for electrical connection to the electronic control device. An elastomer such as silicone 130 is preferably used to support and protect the lead, including the cable and connection elements, as shown in FIG. 9A.

FIG. 9B shows a perspective view of the implantable medical assembly having double-sided terminal ends which can be connected to a test device or stimulation source (not shown). If a double-sided connection portion is required, the end portions of layer can be wrapped around a layer of silicon elastomer 130 having a certain radius to minimize conduction circuit breaks. Then, this portion can be constrained with silicone 130.

The present invention may be applied to the electrical connection (lead or cable) between implantable housings or medical devices in which electronic circuits reside. That is, in any implantable medical device designed to deliver or receive electrical signals, the present invention ensures safe and reliable delivery or receipt of those electrical signals. Further, this implantable medical assembly can be applied to the electrical connection between an implantable housing and an implantable antenna for RF communication used in an implantable medical device.

Moreover, as described above, it is seen that the implantable medical assembly described herein may be manufactured using low cost technology and simple-to-implement manufacturing techniques for mass production.

Finally, it is seen that the implantable medical assembly of the present invention may be safely and reliably used in various nerve stimulation assemblies.

The above descriptions are intended to illustrate the preferred and alternative embodiments of the invention. It will be appreciated that modifications and adaptations to such embodiments may be practiced without departing from the scope of the invention, such scope being most properly defined by reference to this specification as a whole and to the following claims.

1. A method of fabricating a neurostimulation circuit comprising:
providing at least two stimulation assemblies; and
stacking said assemblies to form a single structure;
wherein each of said assemblies has at least one electrode and at least one conductor embedded in a thermoformable insulator, and wherein the surface of said electrode is exposed by removing an area of said thermoformable insulator.

2. The method of fabricating a neurostimulation circuit of claim 1 wherein said thermoformable insulator is made of biocompatible polymer.

3. The method of fabricating a neurostimulation circuit of claim 2 wherein said stimulation assemblies are melted and then conglomerated to form said single structure by heat-treatment.

4. The method of fabricating a neurostimulation circuit of claim 2 wherein said stimulation assemblies can be stacked together with adhesive.

5. The method of fabricating a neurostimulation circuit of claim 1 wherein said electrode and conductor are made of a material selected from among titanium, platinum, tantalum, niobium, iridium, gold or alloys thereof.

6. The method of fabricating a neurostimulation circuit of claim 1 wherein said stimulation assemblies are stacked in an offset fashion, thereby exposing each of said electrodes on said assemblies.

7. The method of fabricating a neurostimulation circuit of claim 3 wherein said single structure or a portion thereof can be over molded with silicone.

8. A neurostimulation circuit to provide a neurostimulation signal to a human nerve comprising:
at least two neurostimulation assemblies which are stacked to form a single structure,
wherein each of said assemblies has at least one electrode and at least one conductor embedded in a thermoformable insulator,
and wherein the surface of said electrode is exposed by removing said thermoformable insulator.

9. The neurostimulation circuit of claim 8 wherein said thermoformable insulator is made of biocompatible polymer.

10. The neurostimulation circuit of claim 9 wherein said stimulation assemblies are melted and then conglomerated to form said single structure by heat-treatment.

11. The neurostimulation circuit of claim 9 wherein said stimulation assemblies can be stacked together with adhesive.

12. The neurostimulation circuit of claim 8 wherein said electrode and conductor are made of a material selected from among titanium, platinum, tantalum, niobium, iridium, gold or alloys thereof.

13. The neurostimulation circuit of claim 8 wherein said stimulation assemblies are stacked in an offset fashion, thereby exposing each of said electrodes on said assemblies.

14. The neurostimulation circuit of claim 10 wherein said single structure or a portion thereof can be over molded with silicone.

15. The neurostimulation circuit of claim 8, wherein said neurostimulation circuit comprises part of an implantable medical assembly

16. The neurostimulation circuit of claim 15, wherein said implantable medical assembly is a cochlear implant.

17. The method of fabricating a neurostimulation circuit of claim 4 wherein said single structure or a portion thereof can be over molded with silicone.

18. The neurostimulation circuit of claim 11 wherein said single structure or a portion thereof can be over molded with silicone.
